(19) United States
(12) Patent Application Publication
(10) Pub. No.: US 2008/0146498 A1
(43) Pub. Date: Jun. 19, 2008

(54) Diagnostic Agent For Ischemic Heart Disease Risk Group

(75) Inventors: Masao Daimon, Chiba (JP); Tohru Minamino, Chiba (JP); Kenji Hashimoto, Tokyo (JP); Issei Komuro, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignees: Masao Daimon, Yotsukaido-shi (JP); Daiichi Pure Chemicals Co., Ltd., Chuo-ku (JP)

(21) Appl. No.: 10/591,604

(22) PCT Filed: Mar. 2, 2005
(86) PCT No.: PCT/JP05/03500

§ 371 Date: Aug. 30, 2007

(30) Foreign Application Priority Data
Mar. 3, 2004 (JP) 2004-059065

(51) Int. Cl. A61K 38/17; C07K 16/18; A61K 45/00; A61P 9/10; G01N 33/53

(52) U.S. Cl. 514/12; 530/387.1; 435/7.1; 514/789

(57) ABSTRACT

The present invention relates to a diagnostic agent for an ischemic heart disease risk group comprising an anti-brain-derived neurotrophic factor antibody as an effective ingredient, to an assay method for an ischemic heart disease risk group performed by measuring a brain-derived neurotrophic factor concentration in blood, and to a suppressive/preventive drug for ischemic heart disease, particularly for post-infarction myocardial remodeling, comprising a brain-derived neurotrophic factor.

### TECHNICAL FIELD

The present invention relates to a diagnostic agent, a diagnostic method, a prognostic prediction method, and a therapeutic drug, for an ischemic heart disease risk group.

### BACKGROUND ART

The ischemic heart disease caused by coronary arteriosclerosis accounts for approximately 7 to 8% in the total death rate of all diseases in Japan and is a disease that still affects more than 1 million people in the country. The number of patients with this disease is year by year on the rise, largely because of the westernization of diet. The prevention and control of ischemic heart disease are greatly important from the viewpoint of medical economy. Some elements related to our life, such as diabetes mellitus, smoking, hypertension, hyperlipemia, family history, aging, obesity, are known as risk factors for coronary arteriosclerosis. In recent years, metabolic syndromes are also counted among such risk factors, whose chief complaints are known to be insulin resistance, obesity, hypertension, impaired glucose tolerance and the like. These factors cause atherosclerosis in the lumen of the coronary artery supplying nutrients to the heart, leading to serious damage to blood flow. Almost no subjective symptom is felt at the early stage, but the attack of angina pectoris could occur afterward. However, it is known that 30 to 40% of them suffer from asymptomatic ischemic heart disease, which exhibits no symptom even after their conditions grow much more serious. The coronary atheroma, when disrupted by various stresses, forms thrombosis in the coronary artery lumen and thereby causes a blockage, leading to acute myocardial infarction. The progression of therapeutic methods, such as reperfusion therapy, contributed to reduction of the death rate of acute myocardial infarction. However, the death rate at the acute stage amounts to nearly 30%, and its main causes are arrhythmia, pump failure, cardiac rupture and the like, associated with myocardial necrosis. Even after the acute stage, myocardial remodeling could take place due to the necrosis of cardiac muscle, resulting in reduced cardiac function. At the chronic stage, cardiac failure, arrhythmia and the like, caused by the above factors, are the causes of death, and therefore, strict drug therapies and regimens become important. The prognostic prediction thereof is crucial in determining the strategies of the drug therapies and regimens. Now that recurrence is observed in approximately 30% of myocardial infarction cases, the control of risk factors is becoming important as described above.

The importance of screening for an ischemic heart disease risk factor group, control of the risk factors, early diagnosis, treatment including myocardial protection, prognostic prediction methods after affection, and recurrence prevention has been recognized from such conditions of the disease. In addition to the measurement of serum cholesterol levels, blood sugar levels, and blood pressure and to the listening to smoking history, hypo-HDL cholesterolemia (see Non-Patent Document 1), insulin resistance (see Non-Patent Document 2), hyperhomocysteinemia (see Non-Patent Document 3), oxidized LDL (see Non-Patent Document 4), and so on, in relation to ischemic heart disease are known as items of screening for an ischemic heart disease risk factor group. Accordingly, the measurement of serum concentrations thereof has been practiced in the screening. A nuclear medicine approach using nuclide tracers is currently used as a main prognostic prediction method. In addition to antiplatelet drugs and coronary dilators, angiotensin converting enzyme inhibitors (see Non-Patent Document 5) and so on are used in the drug therapies. However, the screening and treatment of an ischemic heart disease risk factor group remain insufficient. Moreover, the nuclear medicine approach for prognostic prediction places an economic burden and is limited by facilities capable of practicing it.

A brain-derived neurotrophic factor (hereinafter, referred to as BDNF) is one of neurotrophic factors found in the brain and has been shown to play a key role in the formation or development of neural networks in the brain and further in the preservation of the existence thereof. In the latter half of the 1990s, BDNF was also found to participate in synaptic plasticity and play a key role in memory and learning, and further reported to have neuroprotective action or neuron death. Recently, this BDNF has further been reported to play a key role in not only the nervous system but the cardiovascular system. Recent studies using genetically modified animals have suggested that BDNF is important for the stability of coronary endothelial cells and is involved in vascularization (see Non-Patent Document 6). Moreover, it has also been reported that the vascular endothelium itself synthesizes and secretes BDNF, and that the BDNF synthesis and expression are promoted by angiopathy and myocardial ischemia (see Non-Patent Documents 7 and 8). In addition, it has been suggested that BDNF is likely to protectively work against vascular endothelial damage caused by hyperlipemia (see Non-Patent Document 9), and that decreased BDNF levels are likely to deteriorate the symptoms of metabolic syndrome that exhibits impaired glucose tolerance, lipid metabolism abnormality, or the like and promotively works for arteriosclerosis (see Non-Patent Document 10). However, the role of BDNF in ischemic heart disease, particularly the relationship thereof with myocardial remodeling after acute myocardial infarction has not been reported.

[Non-Patent Document 1] Atheroscler. Tromb. Vasc. Biolo. (1995) 15: 431-440

[Non-Patent Document 2] Diabetes (1988) 37: 1595-1607

[Non-Patent Document 3] JAMA (1992) 268: 877-881

[Non-Patent Document 4] J. Clin. Invest. (1991) 88: 1785-1792

[Non-Patent Document 5] Eur. Heart. J. (1998) 19: A12-A19

[Non-Patent Document 6] Development (2000) 127: 4531-4540

[Non-Patent Document 7] FASEB (2000) 470: 113-117

[Non-Patent Document 8] Journal of pathology (2001) 194: 247-253

[Non-Patent Document 9] Arch. Physiol. Biochem. (2001) 109: 357-360

[Non-Patent Document 10] J. Urol. (2003) 169: 1577-1578

### DISCLOSURE OF THE INVENTION

Ischemic heart disease, as described above, is one of leading killers in Japan and is a disease that still affects increasing numbers of patients. This disease asymptomatically progresses in many cases, and its screening or therapeutic method is still not sufficient. Moreover, a prognostic prediction method for determining therapeutic strategies after affection is not sufficient, either. Thus, there has been growing demand for the development of a diagnostic agent capable of early diagnosis of risk factors for ischemic heart disease, a diagnostic method thereof, a therapeutic drug thereof, and a convenient prognostic prediction method thereof.

The present inventors have conducted diligent studies for solving the problems and have consequently found out that a serum BDNF level is significantly decreased in patients with ischemic heart disease as compared with that in normal individuals, and that the use of this difference allows for the diagnosis of an ischemic heart disease risk group by measuring BDNF by use of an anti-brain-derived neurotrophic factor antibody (hereinafter, referred to as an “anti-BDNF antibody”). The present inventors have also found out that the treatment of ischemic heart disease, particularly the suppression of post-infarction myocardial remodeling, can be achieved by administering BDNF or a drug increasing BDNF. The present invention has been completed based on these findings.

Namely the present invention provides inventions with aspects described below.

1. A diagnostic agent for an ischemic heart disease risk group comprising an anti-BDNF antibody.

2. The diagnostic agent for an ischemic heart disease risk group according to 1, wherein the diagnostic is intended for the measurement of a BDNF concentration in blood.

3. The diagnostic agent for an ischemic heart disease risk group according to 1 or 2, wherein the diagnostic agent comprises an anti-BDNF antibody and a labeled anti-BDNF antibody.

4. A diagnostic kit for an ischemic heart disease risk group comprising an anti-BDNF antibody and a labeling agent.

5. The diagnostic kit for an ischemic heart disease risk group according to 4, wherein the diagnostic kit is intended for the measurement of a BDNF concentration in blood.

6. The diagnostic kit for an ischemic heart disease risk group according to 4 or 5, wherein the diagnostic kit comprises an anti-BDNF antibody and a labeled anti-BDNF antibody.

7. An assay method for an ischemic heart disease risk group, characterized by comprising measuring a BDNF concentration in blood.

8. The assay method for an ischemic heart disease risk group according to 7, wherein the assay method measures a BDNF concentration by use of an anti-BDNF antibody.

9. The assay method for an ischemic heart disease risk group according to 7, wherein the assay method measures a BDNF concentration by use of an anti-BDNF antibody and a labeled anti-BDNF antibody.

10. An assay method for a therapeutic drug for ischemic heart disease, characterized by comprising measuring a BDNF concentration in blood.

11. A therapeutic drug for ischemic heart disease comprising a compound increasing BDNF.

12. A therapeutic drug for ischemic heart disease comprising BDNF.

13. Use of a compound increasing BDNF for the production of a therapeutic drug for ischemic heart disease.

14. Use of BDNF for the production of a therapeutic drug for ischemic heart disease.

15. A therapeutic method for ischemic heart disease, characterized by comprising administering a compound increasing BDNF.

16. A therapeutic method for ischemic heart disease, characterized by comprising administering BDNF.

17. A suppressive/preventive drug for post-infarction myocardial remodeling comprising BDNF.

18. Use of BDNF for the production of a suppressive/preventive drug for post-infarction myocardial remodeling.

19. A suppressive/preventive method for post-infarction myocardial remodeling, characterized by comprising administering BDNF.

By use of the diagnostic agent for an ischemic heart disease risk group of the present invention, an ischemic heart disease risk group such as coronary arteriosclerosis, angina pectoris, and acute and old myocardial infarction can be diagnosed accurately by measuring BDNF in blood. The diagnosis is easily performed by measuring a BDNF concentration in patient's blood, particularly by use of an anti-BDNF antibody and a labeled anti-BDNF antibody. According to the present invention, a therapeutic drug for ischemic heart disease comprising BDNF or a compound increasing BDNF, and a suppressive/preventive drug for post-infarction myocardial remodeling are provided.

### BRIEF DESCRIPTION OF THE DRAWINGS

FIG. 1 is a scatter diagram of a serum BDNF concentration in normal controls (NC) and patients with ischemic heart disease (IHD);

FIG. 2 is a scatter diagram of a serum BDNF concentration in diabetes mellitus-complicated cases (DM(+)) and diabetes mellitus-noncomplicated cases (DM(−)) of patients with ischemic heart disease;

FIG. 3 shows the correlation between a serum BDNF concentration and a blood sugar (BS) level in patients with ischemic heart disease;

FIG. 4 shows the correlation between a serum BDNF concentration and a glycohemoglobin (HbA1c) level in patients with ischemic heart disease;

FIG. 5 is a scatter diagram of a serum BDNF concentration in hyperlipemia-complicated cases (HL(+)) and hyperlipemia-noncomplicated cases (HL(−)) of patients with ischemic heart disease;

FIG. 6 shows the correlation between a serum BDNF concentration and a serum total cholesterol (T-cho) level in patients with ischemic heart disease;

FIG. 7 shows the correlation between a serum BDNF concentration and a serum LDL cholesterol (LDL) level in patients with ischemic heart disease;

FIG. 8 is a scatter diagram of a serum BDNF concentration in hypertension-complicated cases (HT(+)) and hypertension-noncomplicated cases (HT(−)) of patients with ischemic heart disease;

FIG. 9 shows the correlation between a serum BDNF concentration and systolic blood pressure in patients with ischemic heart disease;

FIG. 10 shows the correlation between a serum BDNF concentration and diastolic blood pressure in patients with ischemic heart disease;

FIG. 11 is a scatter diagram of a serum BDNF concentration in smoking cases (smoking (+)) and nonsmoking cases (smoking (−)) of patients with ischemic heart disease;

FIG. 12 shows the difference of a serum BDNF concentration among patients with ischemic heart disease according to CCS scores;

FIG. 13 shows a protocol of myocardial infarction model construction;

FIG. 14 shows macroscopic findings on heart specimens;

FIG. 15 shows a result of Masson trichrome stain of heart slices; and

FIG. 16 shows the size of myocardial infarction (Masson trichrome stain).

### BEST MODE FOR CARRYING OUT THE INVENTION

Hereinafter, a diagnostic agent for an ischemic heart disease risk group, diagnostic kit, assay method for an ischemic heart disease risk group, therapeutic drug for ischemic heart disease, and assay method thereof of the present invention will be described in detail.

The meanings or definitions of terms used herein are as follows:

An “anti-brain-derived neurotrophic factor antibody (anti-BDNF antibody)” refers to an antibody obtained by using BDNF as an antigen. The antibody may have the ability to bind to BDNF and includes polyclonal antibodies, monoclonal antibodies, and antibodies obtained by gene recombination techniques. If necessary, the antibody may be purified or modified chemically or may be fragmented such as F(ab′)₂. A preferable anti-BDNF antibody includes polyclonal and monoclonal antibodies that specifically bind to BDNF. A commercially available product can be used as the anti-BDNF monoclonal antibody.

A “labeled anti-brain-derived neurotrophic factor antibody (labeled anti-BDNF antibody)” refers to an antibody engineered so that BDNF in a sample can be detected by binding and labeling the anti-BDNF antibody with an enzyme (e.g., peroxidase, β-D-galactosidase, alkaline phosphatase, and glucose-6-phosphate dehydrogenase), fluorescent material, radioisotope or isotope, gold colloid particle, color latex, or the like. The “labeled anti-BDNF antibody” further includes anti-BDNF antibodies modified with biotin, 2,4-dinitrophenol, or the like. For using these anti-BDNF antibodies modified with biotin, 2,4-dinitrophenol, or the like, BDNF in a sample can be detected with high sensitivity by using the labeled anti-BDNF antibody together with labeled avidin or a labeled anti-2,4-dinitrophenol antibody.

“Ischemic heart disease” includes coronary arteriosclerosis, angina pectoris, and acute and old myocardial infarction and refers to the serious disorder of the vital heart that mostly affects males and females in late middle age or older. Coronary arteriosclerosis is characterized by arteriosclerosis in the coronary artery that supplies nutrients to the heart. Angina pectoris is characterized by attacks of chest pain caused by impaired blood flow in the coronary artery. Myocardial infarction is characterized by myocardial necrosis caused by impaired blood flow in the coronary artery and by fatal complications coming there with such as arrhythmia, cardiac failure, cardiac rupture, and pump failure. Impaired blood flow to the heart, a vital organ, is an essential characteristic of these ischemic heart diseases.

“Post-infarction myocardial remodeling” refers to a series of changes such as the hypertrophy of myocardial cells at non-infarction sites, increase in interstitial tissue (extracellular matrix), and the dilation of cardiac lumens, which occur in compensation for reduced cardiac function caused by thickening at infarction sites after myocardial infarction. Since long-term prognosis after myocardial infarction is correlated with the degree of left ventricular dysfunction, the suppression of myocardial remodeling is important for maintaining and conserving the function of the left ventricle.

The diagnosis of an ischemic heart disease risk factor group according to the present invention can be performed by measuring a BDNF level, for example in human blood. Specifically, serum is prepared from human blood to measure a serum BDNF level by a variety of methods. Preferably, the measurement of a BDNF level in blood is performed by immunological methods using an anti-BDNF antibody, more preferably immunological methods using an anti-BDNF antibody and a labeled anti-BDNF antibody. It is desirable that BDNF should be detected and quantified by sandwich ELISA using an antibody having high specificity for BDNF.

To be more specific, a method is preferable in which a serum sample is brought into contact with an anti-BDNF antibody immobilized on a solid phase, and after the washing of the solid phase, a labeled anti-BDNF antibody is brought into contact therewith to measure a BDNF level by use of the label. In this context, the labeled anti-BDNF antibody includes an anti-BDNF antibody labeled with a directly measurable labeling material, the combination of biotin and avidin, and the combination of 2,4-dinitrophenol and an antibody against it, as described above.

A specific method for measuring BDNF in blood includes a method comprising the steps of:

1. immobilizing an anti-BDNF antibody onto a solid phase such as polystyrene, nylon, glass, silicon rubber, and Sepharose;

2. adding to the solid phase or contacting with the solid phase serum collected from a patient to be diagnosed;

3. washing the solid phase;

4. adding thereto or contacting therewith a labeled anti-BDNF antibody; and

5. measuring a BDNF level by use of the label.

Furthermore, a specific method for measuring BDNF in serum includes a method comprising the following steps

1. immobilizing an anti-BDNF antibody onto a solid phase such as polystyrene, nylon, glass, silicon rubber, and Sepharose;

2. adding to the solid phase or contacting with the solid phase serum collected from a patient to be diagnosed;

3. washing the solid phase;

4. adding thereto or contacting therewith an anti-BDNF antibody modified with biotin or 2,4-dinitrophenol;

5. adding thereto or contacting therewith labeled avidin or a labeled 2,4-dinitrophenol antibody; and

6. measuring a BDNF level by use of the label.

The specific method for measuring BDNF in serum further includes a method comprising the steps of:

1. immobilizing an anti-BDNF antibody onto a solid phase such as polystyrene, nylon, glass, silicon rubber, and Sepharose;

2. adding to the solid phase or contacting with the solid phase blood collected from a patient to be diagnosed;

3. washing the solid phase;

4. adding thereto or contacting therewith an anti-BDNF antibody modified with biotin;

5. adding thereto or contacting therewith labeled avidin; and

6. measuring a BDNF level by use of the label.

The form of the solid phase includes pellets, wells, test tubes, and membranes such as nitrocellulose.

A commercially available product can be used as BDNF used as an antigen or ELISA standard. Alternatively, the BDNF can be purified from biological materials or prepared by a genetic engineering approach. When the genetic engineering approach is used, a gene encoding BDNF is incorporated into an appropriate vector, which is in turn inserted and transformed into an appropriate host. As a result, recombinant BDNF of interest can be obtained from the culture supernatant of this transformation. This approach is suitable for the homogeneous and large-scale production of BDNF. The host cell is not particularly limited, and a variety of host cells conventionally used in genetic engineering approaches, for example Escherichia coli, Bacillus subtilis, yeast, plant, or animal cells, can be used.

The anti-BDNF antibody is prepared by immunizing mice, rats, rabbits, chickens, turkeys, horses, goats, or the like, with BDNF used as an antigen. The labeled anti-BDNF antibody can be prepared by a routine method or by use of a commercially available biotinylation reagent or peroxidase with a crosslinking agent.

The diagnosis of an ischemic heart disease risk group can be performed by establishing certain criteria for a BDNF concentration in blood and comparing and evaluating the measured BDNF concentration of a blood sample with the criteria. A method for establishing the criteria includes a method in which 95 percentile values or ROC curves frequently used in the field of clinical examination are used to establish criteria from desired examination precision. A BDNF concentration in the blood of a patient with ischemic heart disease is significantly lower than that in a normal individual. Therefore, the risk of ischemic heart disease can be diagnosed to be high by measuring a BDNF level in blood by the means described above and comparing the BDNF level with that in a normal individual.

Such evaluation includes a method performed by BDNF measurement alone and a method performed by linking the measurement value of BDNF to the measurement values of other indices, for example makers known in the art. In this context, the term “linking” refers to using calculation formula to obtain information that can not be obtained in BDNF measurement alone. A method for the linking can be exemplified by a method in which the measurement value of BDNF is divided by the measurement value of the marker known in the art and using the determined ratio (ratio between these two measurement values) as a new index. As a result, the precision of examination/diagnosis can be adjusted as desired.

The diagnostic agent or diagnostic kit for an ischemic heart disease risk group of the present invention may contain an anti-BDNF antibody, anti-BDNF antibody and a labeling agent, or an anti-BDNF antibody and a labeled anti-BDNF antibody.

A method of the present invention is also useful in the assay of a therapeutic drug for ischemic heart disease. Namely, the method can assay the therapeutic effect of a therapeutic drug for ischemic heart disease. Moreover, a compound having the action of increasing BDNF is useful as a therapeutic drug for ischemic heart disease. Model animals (e.g., mice and rats) with low BDNF levels are also useful as animal models of ischemic heart disease. Thus, the use of this assay method allows for the screening of a novel therapeutic drug for ischemic heart disease.

The therapeutic drug found by such a method can include drugs that can be administered parenterally or orally. The therapeutic drug for ischemic heart disease is exemplified by, in addition to BDNF itself, an azole derivative represented by the following formula (1) (Japanese Patent Laid-Open No. 2001-131161):

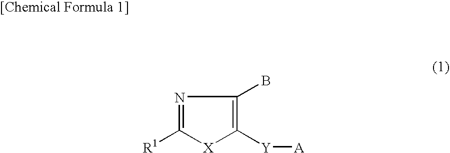

(wherein R¹ represents a halogen atom, a heterocyclic group which may be substituted, a hydroxy group which may be substituted, a thiol group which may be substituted, or an amino group which may be substituted; A represents an acyl group which may be substituted, a heterocyclic group which may be substituted, a hydroxy group which may be substituted, or a carboxyl group which may be esterified or amidated; B represents an aromatic group which may be substituted; X represents an oxygen atom, a sulfur atom, or a nitrogen atom which may be substituted; and Y represents a divalent hydrocarbon group or a heterocyclic group).

Alternatively, it is exemplified by a 5-phenylpyrimidine compound represented by the following formula (2) and a salt thereof (Japanese Patent Laid-Open No. 8-3142):

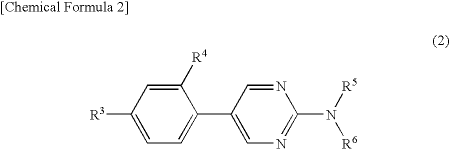

(wherein each of R³ and R⁴ is an halogen atom; and each of R⁵ and R⁶ is a hydrogen atom, an alkyl group having 1 to 5 carbon atoms, or an alkylsulfonyl or acetylaminoalkyl group having 1 to 3 carbon atoms).

It further includes catechol derivatives (Furukawa. Y., J. Biol. Chem., Vol. 261, pp. 6039 (1986); and Japanese Patent Laid-Open Nos. 63-83020, 63-156751, 2-53767, 2-104568, 2-149561, 3-99046, 3-83921, 3-86853, and 5-32646), quinine derivatives (Japanese Patent Laid-Open Nos. 3-81218, 4-330010, and 7-285912), glutamic acid derivatives (Japanese Patent Laid-Open No. 7-228561), unsaturated fatty acid derivatives (Japanese Patent Laid-Open No. 8-143454), eudesmane derivatives (Japanese Patent Laid-Open No. 8-73395), annelated oxazole derivatives (Japanese Patent Laid-Open No. 8-175992), carbazole derivatives (Japanese Patent Laid-Open No. 8-169879), indole derivatives (Japanese Patent Laid-Open Nos. 7-118152 and 8-239362), natural product-derived terpene derivatives (Japanese Patent Laid-Open Nos. 7-149633 and 8-319289), and a purine derivative leteprinim (NeuroTherapeutics, US).

Of these compounds, 2-amino-S-(2,4-dichlorophenyl)pyrimidine (Biochemical Pharmacology 66 (2003) 1019-1023) and 4-(4-chlorophenyl)-2-(2-methyl-1H-imidazol-1-yl)-5-[3-(2-methoxyphenoxy)propyl]-1,3-oxazole (Chem. Pharm. Bull. 51 (5) 565-573 (2003)) are preferable.

The accurate doses and dosage schedules of these therapeutic drugs for ischemic heart disease differ depending on a requirement for each individual to be treated, a therapeutic method, the degree of disease, the degree of necessity, and the type of the drug, and are naturally required to be selected at a physician's discretion. For example, the dose and number of doses of BDNF parenterally administered differ depending on symptoms, age, body weight, dosage form, and so on. For example when BDNF is hypodermically or intravenously administered as an injection, the dose thereof in adult patient is selected from among the range of approximately 0.1 to approximately 2500 mg/kg of body weight/day, preferably approximately 1 to approximately 500 mg/kg of body weight/day. For example when BDNF is administered as a spray into the trachea, the dose thereof in adult patient is selected from among the range of approximately 0.1 to approximately 2500 mg/kg of body weight/day, preferably approximately 1 to approximately 500 mg/kg of body weight/day. The dosage schedule is daily administration, intermittent administration, or a combination thereof. The dose and number of doses of BDNF orally administered differ depending on symptoms, age, body weight, dosage form, and so on. For example, the dose thereof in adult patient is selected from among the range of approximately 0.5 to approximately 2500 mg/kg of body weight/day, preferably approximately 1 to approximately 1000 mg/kg of body weight/day.

The therapeutic drug for ischemic heart disease of the present invention can be mixed with a pharmacologically acceptable nontoxic carrier to produce a pharmaceutical composition. When such a composition is prepared for parenteral administration (hypodermic injection, intramuscular injection, or intravenous injection), a particularly preferable dosage form thereof is a solution or suspension. When the composition is prepared for vaginal or rectal administration, a particularly preferable dosage form thereof is a semisolid dosage form such as a cream or suppository. When the composition is prepared for nasal administration, a particularly preferable dosage form thereof is a powder, drop for nose, or aerosol.

The composition can be administered in a single-dose dosage form and can be prepared by any method well known in pharmaceutical techniques as described in, for example Remington's Pharmaceutical Sciences (Mack Publishing Company, Easton, Pa., 1970). A preparation for injection can be supplemented with, for example plasma-derived proteins such as albumin, amino acids such as glycine, or sugars such as mannitol, as a pharmaceutical carrier. When the composition is used in an injection dosage form, a buffer, solubilizing agent, isotonic agent, and so on can further be added thereto. Alternatively, when the composition is used as a water-soluble preparation or freeze-dried preparation, the addition of surfactants such as Tween 80 (registered trademark) and Tween 20 (registered trademark) is preferable for preventing aggregation. Parenteral dosage forms other then an injection may contain distilled water or physiological saline, polyalkylene glycol such as polyethylene glycol, oil of plant origin, hydrogenated naphthalene, and so on. For example a preparation for vaginal or rectal administration such as a suppository contains, for example polyalkylene glycol, Vaseline, cacao butter, as general excipients. The preparation for vaginal administration may contain absorption promoters such as bile salt, ethylenediamine salt, and citrate. A preparation for inhalation may be solid and may contain, for example lactose as an excipient. A drop for nasal administration may be an aqueous or oil solution.

### EXAMPLES

Hereinafter, the present invention will be described more fully with reference to Examples of the present invention. However, the present invention is not limited to these Examples.

### Example 1

### (1) Subjects

Subjects selected were 39 patients with ischemic heart disease (29 males and 10 females, average age: 65.0 year old (standard deviation 9.4), age range: 34 to 82 year old) shown in Table 1 below as well as a cohort of 33 normal individuals (11 males and 22 females, average age: 68.3 year old (standard deviation 12.0), age range: 35 to 82 year old) as normal controls. All the patients with ischemic heart disease were subjected to coronary angiography by cardiac catheterization and diagnosed by confirming significant coronary narrowing caused by arteriosclerosis in the coronary artery. According to quantitative assessment in coronary angiography, 50% or more narrowing was defined as significant narrowing. All the test subjects were examined for coronary artery risk factors, that is, their treatment histories for hyperlipemia, diabetes mellitus, and hypertension, and smoking histories. Hyperlipemia was diagnosed by satisfying the diagnostic criteria of Japan Atherosclerosis Society, that is, of serum lipids, total cholesterol not lower than 220 mg/dl, LDL cholesterol not lower than 140 mg/dl, HDL cholesterol less than 40 mg/dl, or triglyceride not lower than 150 mg/dl. Diabetes mellitus was diagnosed by satisfying the diagnostic criteria of Japan Diabetes Society, that is, a fasting venous plasma glucose concentration not lower than 126 mg/dl, a 75-g 2-hour oral glucose tolerance test (OGTT) value not lower than 200 mg/dl, or a random blood sugar level not lower than 200 mg/dl. Hypertension was diagnosed by satisfying the diagnostic criteria of International Society of Hypertension, that is, systolic blood pressure not lower than 140 mmHg or diastolic blood pressure not lower than 90 mmHg. The subjective symptoms of all the subjects were specified according to CCS (Canadian Cardiovascular Society) classification. The CCS classification classifies the exertional symptoms of angina pectoris in four tiers according to severity (see e.g., Campeau, L. et al., Grading Angina pectoris. Circulation (1976) 54: 522).

### (2) Test Procedures

Serum samples of the subjects were collected and stored at −80° C. until measurement. A serum BDNF level was measured with a BDNF measurement kit (“BDNF Emax® ImmunoAssay Systems”, Promega, US) according to the manufacturer's instruction. Namely, a 96-well plate was coated with an anti-BDNF monoclonal antibody, followed by incubation at 4° C. for 18 hours. The plate was blocked with a blocking buffer solution at room temperature for 1 hour. After the washing of the plate with a buffer solution, 100 μL of diluted serum was added thereto. Human BDNF (78 to 5000 pg/mL) was added for use as a standard for quantification. After reaction at room temperature for 2 hours, the plate was washed 5 times with a buffer solution. An anti-human BDNF antibody was added thereto and reacted at room temperature for 2 hours. The plate was washed 5 times with a buffer solution. A horseradish peroxidase-labeled anti-IgY antibody (100 μL) was added thereto and reacted at room temperature for 1 hour. The plate was washed 5 times with a buffer solution. A TMB solution (100 μL) was added thereto and reacted at room temperature for 10 minutes. The reaction was terminated by the addition of a stop solution (1 M hydrochloric acid; 100 μL). Within 30 minutes, absorbance at a wavelength of 450 nm was measured with an automatic microplate reader (Emax, Molecular Devices, US). A BDNF content in the samples was measured by sandwich ELISA to calculate a BDNF concentration thereof from a calibration curve. At the same time, all the subjects were measured for their blood pressures, blood sugar levels, glycohemoglobin (HbA_{1c}) levels, serum total cholesterol levels, and serum LDL cholesterol levels.

### (3) Statistical Analysis

Data was indicated in mean value ±standard deviation. The statistical analysis between two groups was performed by use of Student's t-test. The relationship between variables was confirmed by Pearson's product-moment correlation coefficient. The difference among multiple groups was analyzed by one-way analysis of variance (ANOVA). For the multiple comparison among subjective symptoms, Bonferroni/Dunn test was conducted. p values of 0.05 or lower were defined to be statistically significant.

### (4) Result

The characteristics and experimental results of the patients with ischemic heart disease are shown in Table 1.

TABLE 1

| | | | | BDNF | Complication (1: present, 0: absent) | | | | Blood pressure | | | | | | | Subjective |
|---|---|---|---|---|---|---|---|---|---|---|---|---|---|---|---|---|
| | | | | level | Diabetes | Hyper- | Hyper- | Smoking | (mmHg) | | Biochemical examination (mg/dl) | | | | | symptom |
| No. | Age | Sex | Disease | (ng/ml) | mellitus | lipemia | tension | history | SBP | DBP | BS | HbAlc | T-cho | TG | LDL | CCS |
| 1 | 67 | M | AMI | 15.4 | 1 | 1 | 1 | 1 | 120 | 80 | 120 | 4.6 | 141 | 58 | 89 | I |
| 2 | 71 | M | AP | 14.5 | 1 | 1 | 1 | 1 | 148 | 86 | 156 | 6.3 | 169 | 81 | 113 | Absent |
| 3 | 71 | M | OMI | 33.2 | 1 | 1 | 1 | 0 | 130 | 80 | 95 | 5.1 | 134 | 55 | 110 | II |
| 4 | 54 | M | AMI | 41.9 | 1 | 1 | 1 | 1 | 104 | 64 | 78 | 7.2 | 150 | 162 | 88 | I |
| 5 | 74 | M | AP | 13.1 | 1 | 1 | 1 | 1 | 158 | 75 | 205 | 7.2 | 175 | 132 | 106 | I |
| 6 | 70 | F | AP | 5.6 | 1 | 1 | 1 | 0 | 138 | 70 | 199 | 8.2 | 199 | 178 | 133 | I |
| 7 | 59 | M | OMI | 17.8 | 0 | 1 | 1 | 1 | 110 | 66 | 73 | 5.5 | 149 | 122 | 98 | I |
| 8 | 66 | M | OMI | 25.5 | 0 | 1 | 0 | 0 | 130 | 78 | 187 | 5.3 | 166 | 141 | 109 | I |
| 9 | 82 | F | AP | 9.2 | 1 | 1 | 1 | 0 | 142 | 66 | 200 | 6.2 | 148 | 144 | 97 | I |
| 10 | 56 | M | OMI | 27.9 | 1 | 1 | 1 | 1 | 102 | 57 | 88 | 4.2 | 191 | 137 | 126 | Absent |
| 11 | 67 | M | AP | 15.2 | 0 | 1 | 1 | 0 | 158 | 96 | 88 | 5.4 | 202 | 242 | 135 | I |
| 12 | 69 | F | OMI | 29.2 | 0 | 1 | 1 | 0 | 106 | 74 | 111 | 5.1 | 205 | 54 | 138 | I |
| 13 | 65 | M | AMI | 22.5 | 0 | 0 | 0 | 1 | 124 | 72 | 116 | 5 | 183 | 134 | 120 | I |
| 14 | 64 | M | AP | 23.7 | 0 | 0 | 0 | 0 | 140 | 88 | 108 | 5.1 | 182 | 112 | 121 | II |
| 15 | 80 | M | AP | 18.9 | 0 | 0 | 0 | 0 | 110 | 69 | 155 | 5.4 | 164 | 150 | 123 | I |
| 16 | 62 | F | AP | 11.8 | 0 | 0 | 1 | 1 | 132 | 80 | 142 | 6 | 237 | 240 | 160 | I |
| 17 | 74 | M | AMI | 19.2 | 0 | 1 | 1 | 1 | 144 | 82 | 124 | 5.2 | 195 | 180 | 126 | I |
| 18 | 77 | F | AP | 29.2 | 1 | 1 | 1 | 0 | 150 | 58 | 140 | 7 | 152 | 106 | 95 | I |
| 19 | 68 | M | OMI | 16.9 | 0 | 1 | 0 | 1 | 100 | 62 | 86 | 4.7 | 164 | 111 | 107 | I |
| 20 | 48 | M | AMI | 2 | 0 | 0 | 0 | 0 | 106 | 68 | 90 | 4.8 | 201 | 147 | 132 | I |
| 21 | 68 | F | AP | 24.9 | 0 | 1 | 0 | 0 | 156 | 76 | 114 | 5.7 | 215 | 84 | 149 | I |
| 22 | 60 | M | OMI | 8.6 | 0 | 1 | 1 | 0 | 92 | 60 | 117 | 4.8 | 162 | 88 | 109 | II |
| 23 | 55 | M | OMI | 28.1 | 0 | 0 | 0 | 1 | 90 | 60 | 138 | 5.4 | 173 | 125 | 112 | I |
| 24 | 63 | M | OMI | 23.1 | 0 | 0 | 0 | 1 | 89 | 63 | 84 | 4.5 | 206 | 106 | 127 | I |
| 25 | 75 | M | AMI | 18.6 | 0 | 1 | 0 | 1 | 148 | 72 | 122 | 4.7 | 234 | 90 | 151 | II |
| 26 | 63 | M | OMI | 38 | 1 | 1 | 1 | 1 | 134 | 82 | 195 | 6.1 | 208 | 220 | 133 | I |
| 27 | 66 | M | OMI | 18.2 | 0 | 0 | 0 | 1 | 104 | 64 | 99 | 4.7 | 171 | 96 | 107 | I |
| 28 | 60 | M | AP | 23.5 | 0 | 1 | 1 | 1 | 142 | 70 | 107 | 4.9 | 237 | 201 | 153 | I |
| 29 | 69 | M | OMI | 22.9 | 1 | 1 | 0 | 0 | 152 | 80 | 102 | 5.1 | 185 | 84 | 121 | I |
| 30 | 63 | M | OMI | 26.1 | 1 | 0 | 1 | 1 | 136 | 82 | 116 | 6.6 | 127 | 118 | 65 | I |
| 31 | 70 | F | AP | 20.3 | 0 | 1 | 0 | 1 | 108 | 72 | 114 | 5.4 | 190 | 116 | 118 | I |
| 32 | 47 | M | AP | 29.4 | 0 | 1 | 1 | 1 | 140 | 62 | 140 | 4.8 | 209 | 140 | 148 | Absent |
| 33 | 34 | M | OMI | 35.9 | 1 | 1 | 1 | 1 | 110 | 62 | 174 | 5.4 | 112 | 130 | 72 | Absent |
| 34 | 72 | M | AP | 37.6 | 0 | 1 | 1 | 1 | 102 | 76 | 98 | 5.1 | 197 | 190 | 133 | Absent |
| 35 | 78 | F | AP | 22.8 | 0 | 1 | 1 | 0 | 120 | 60 | 99 | 5 | 254 | 64 | 155 | Absent |
| 36 | 59 | M | AMI | 37 | 1 | 0 | 0 | 0 | 102 | 64 | 240 | 7.8 | 169 | 81 | 109 | Absent |
| 37 | 67 | F | OMI | 16.2 | 0 | 1 | 0 | 0 | 136 | 82 | 157 | 5.5 | 159 | 181 | 91 | I |
| 38 | 55 | F | OMI | 7.2 | 0 | 0 | 0 | 0 | 110 | 80 | 138 | 4.6 | 224 | 219 | 146 | I |
| 39 | 63 | M | OMI | 12.7 | 0 | 0 | 0 | 1 | 104 | 59 | 120 | 5.2 | 212 | 342 | 139 | Absent |

AMI; acute myocardial infarction
OMI; old myocardial infarction
AP; angina pectoris
CCS; Canadian Cardiovascular Society

### i) Serum BDNF Concentration in all Subjects

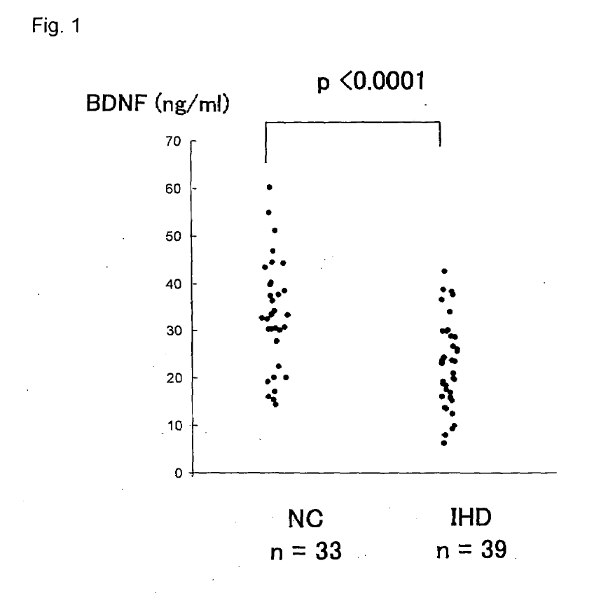

Serum BDNF concentration scattering in the normal controls (NC) and the subjects with ischemic heart disease (IHD) is shown in FIG. 1.

As a result of Student's t-test, the serum BDNF level (mean value: 21.6 ng/mL [standard deviation: 9.6]) in the subjects with ischemic heart disease was shown to be significantly lower than the serum BDNF level (mean value: 33.2 ng/mL [standard deviation: 11.4], p<0.0001) in the normal controls. Considering the NC and IHD groups (n=72) together, no significant correlation (r=−0.120, p=0.317) was observed between the serum BDNF level and age. Considering the NC and IHD groups together, no significant difference was observed between the serum BDNF level (mean value: 25.8 ng/mL [standard deviation: 11.2]) in the males (n=40) and the serum BDNF level (mean value: 28.4 ng/mL [standard deviation: 12.7]) in the female (n=32) (Student's t-test, p=0.347).

### ii) Serum BDNF and Diabetes Mellitus

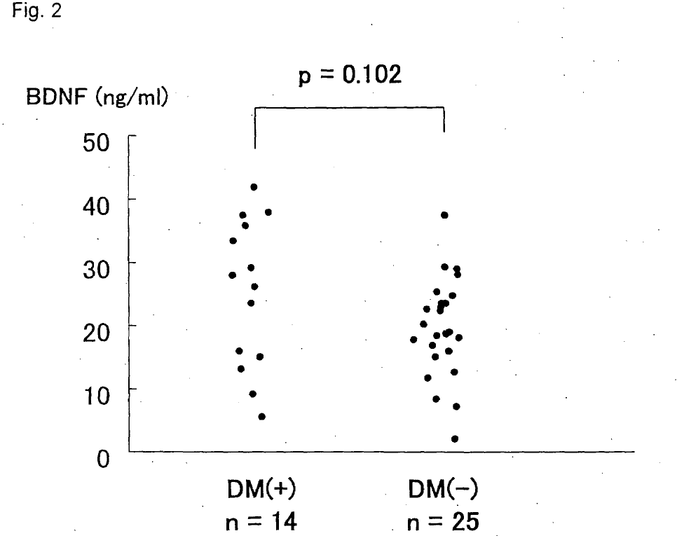

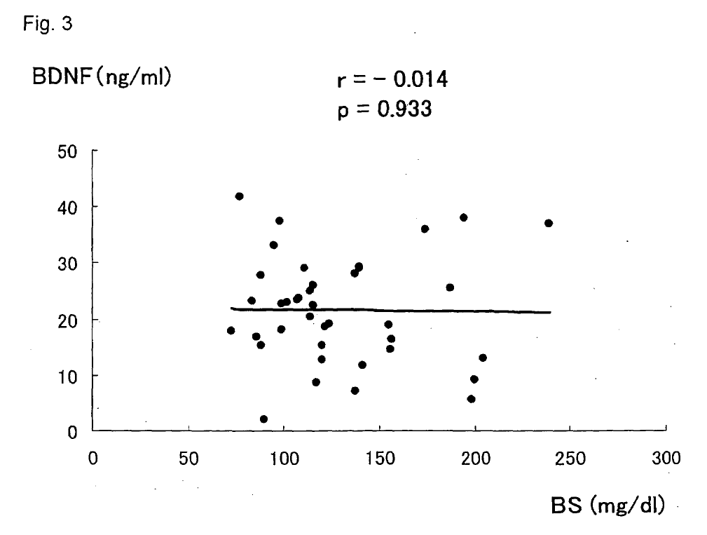

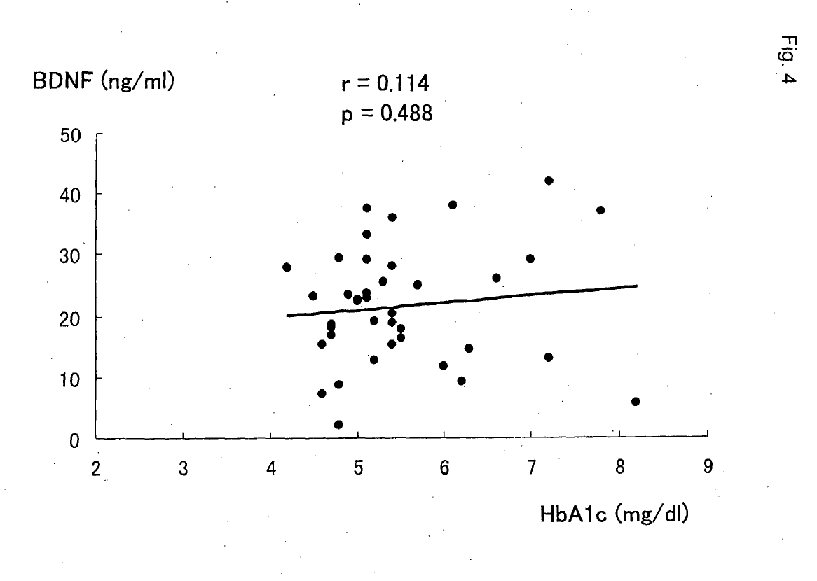

The comparison between the serum BDNF level (mean value: 19.8 ng/mL [standard deviation: 7.7]) of the patients (n=14) with ischemic heart disease having an anamnesis of diabetes mellitus and the serum BDNF level (mean value: 25.0 ng/mL [standard deviation: 11.7]) of the patients (n=25) with ischemic heart disease having no anamnesis of diabetes mellitus is shown in FIG. 2. No significant difference was observed between the serum BDNF levels of these two groups (Student's t-test, p=0.102) The correlation between the serum BDNF levels and blood sugar levels of the patients with ischemic heart disease is shown in FIG. 3, and the correlation between the serum BDNF levels and glycohemoglobin levels of all the subjects is shown in FIG. 4. No significant correlation was observed between the serum BDNF levels and the blood sugar levels (r=−0.014, p=0.933) or the glycohemoglobin levels (r=0.114, p=0.488).

iii) Serum BDNF and Hyperlipemia

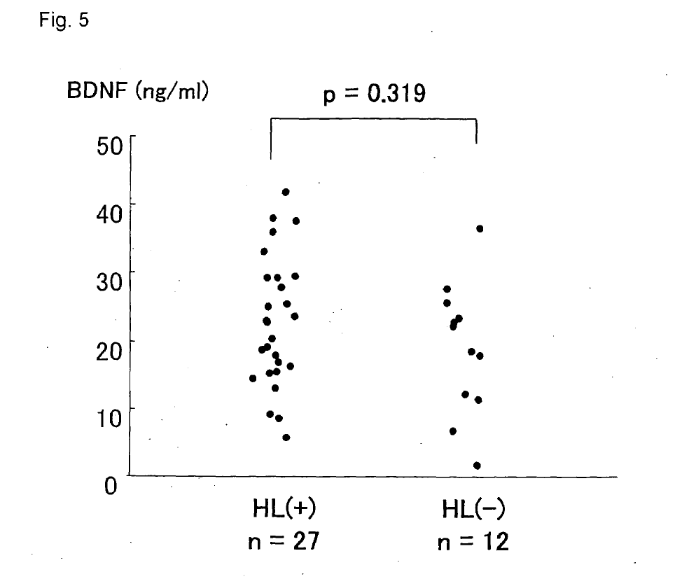

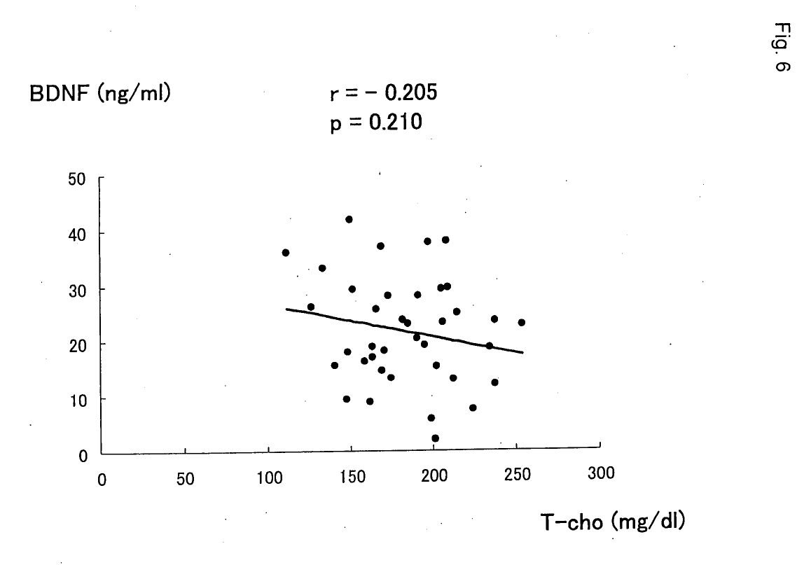

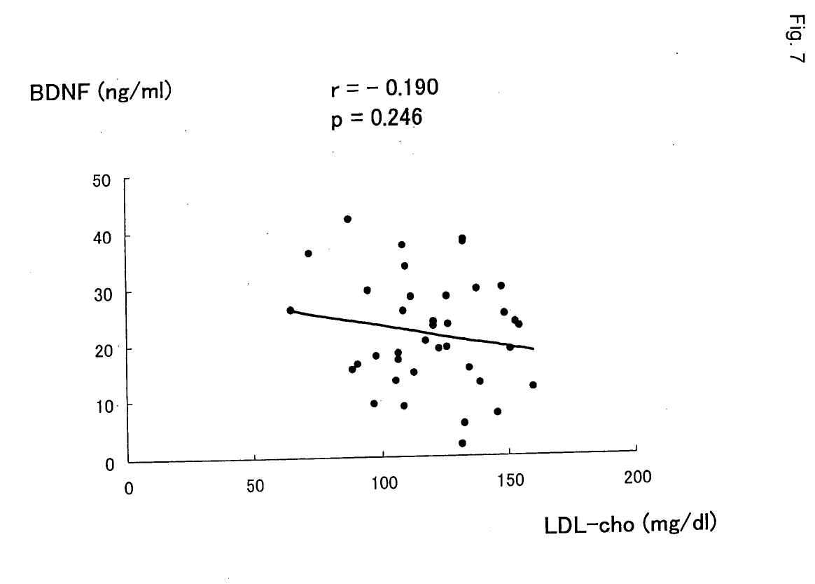

The comparison between the serum BDNF level (mean value: 22.7 ng/mL [standard deviation: 9.5]) of the patients (n=27) with ischemicheart disease having an anamnesis of hyperlipemia and the serum BDNF level (mean value: 19.3 ng/mL [standard deviation: 9.7]) of the patients (n=12) with ischemic heart disease having no anamnesis of hyperlipemia is shown in FIG. 5. No significant difference was observed between the serum BDNF levels of these two groups (Student's t-test, p=0.319) The correlation between the serum BDNF levels and serum total cholesterol levels of the patients with ischemic heart disease is shown in FIG. 6, and the correlation between the serum BDNF levels and serum LDL cholesterol levels of the patients with ischemic heart disease is shown in FIG. 7. No significant correlation was observed between the serum BDNF levels and the serum total cholesterol levels (r=−0.205, p=0.210) or the serum LDL cholesterol levels (r=−0.190, p=0.246).

### iv) Serum BDNF and Hypertension

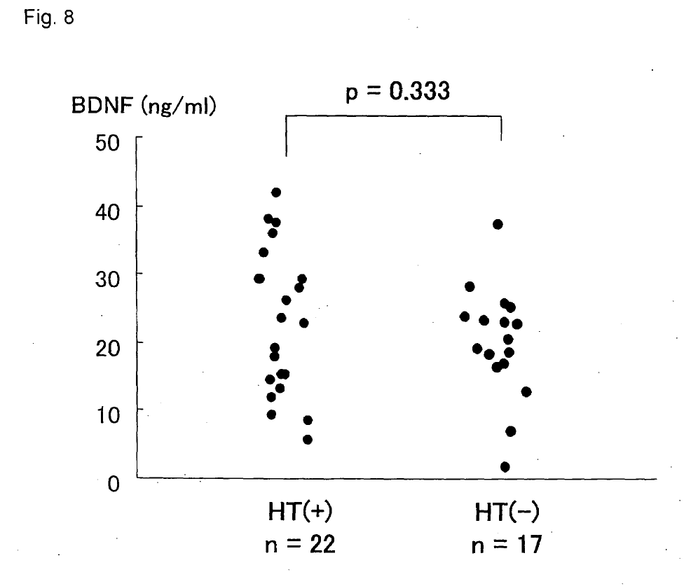

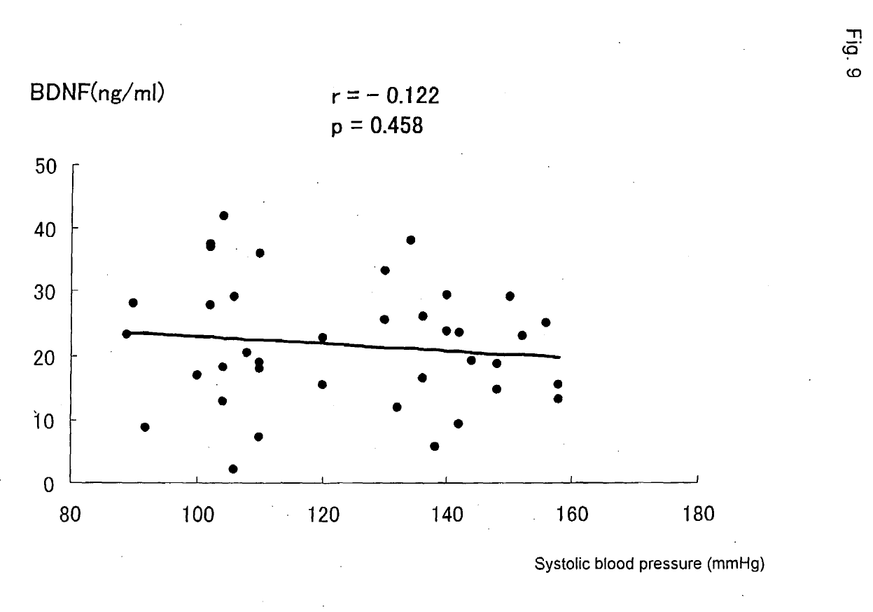

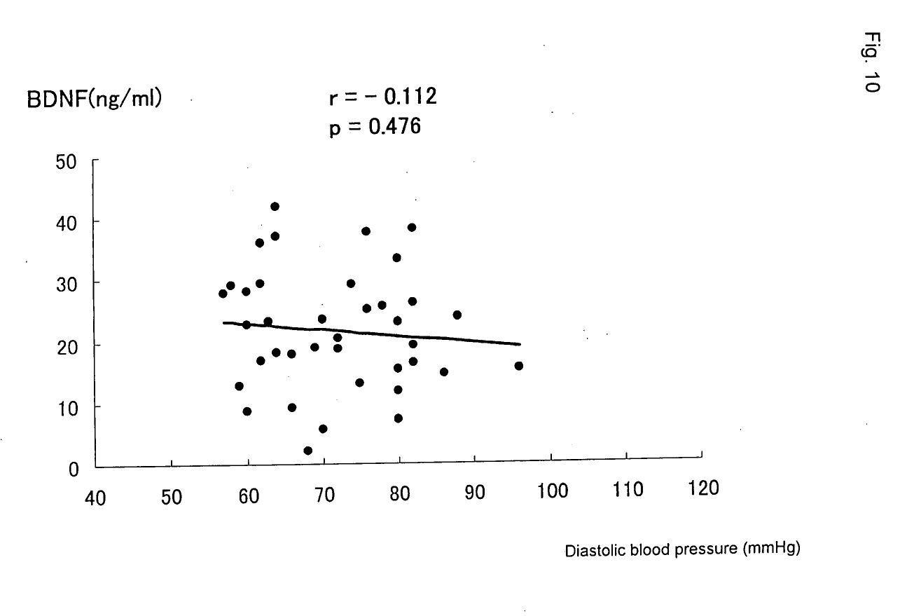

The comparison between the serum BDNF level (mean value: 23.0 ng/mL [standard deviation: 10.6]) of the patients (n=22) with ischemic heart disease having an anamnesis of hypertension and the serum BDNF level (mean value: 19.9 ng/mL-[standard deviation: 8.0]) of the patients (n=17) with ischemic heart disease having no anamnesis of hypertension is shown in FIG. 8. No significant difference was observed between the serum BDNF levels of these two groups (Student's t-test, p=0.333). The correlation between the serum BDNF levels and systolic blood pressures of the patients with ischemic heart disease is shown in FIG. 9, and the correlation between the serum BDNF levels and diastolic blood pressures of the patients with ischemic heart disease is shown in FIG. 10. No significant correlation was observed between the serum BDNF levels and the systolic blood pressures (r=−0.112, p=0.458) or the diastolic blood pressures (r=−0.112, p=0.476).

### v) Serum BDNF and Smoking

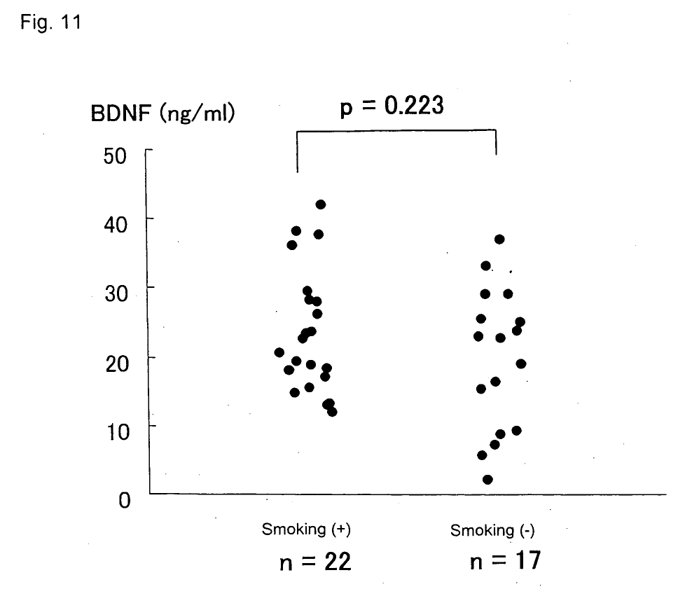

The comparison between the serum BDNF level (mean value: 23.3 ng/mL [standard deviation: 8.9]) of the patients (n=22) with ischemic heart disease having an anamnesis of smoking and the serum BDNF level (mean value: 19.5 ng/mL [standard deviation: 10.3]) of the patients (n=17) with ischemic heart disease having no anamnesis of smoking is shown in FIG. 11. No significant difference was observed between the serum BDNF levels of these two groups (Student's t-test, p=0.223).

### vi) Serum BDNF and Angina Pectoris

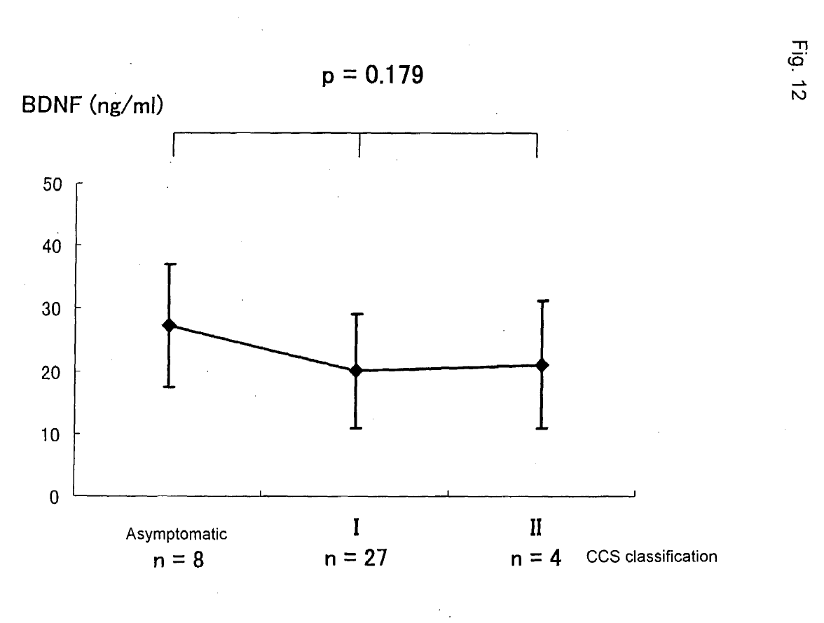

As shown in FIG. 12, no significant difference was observed among the serum BDNF concentrations of the patients with ischemic heart disease classified according to CCS classification (F=1.807, p-0.179).

As seen in the test results, a serum BDNF level was shown to be significantly decreased in the patients with ischemic heart disease as compared with that in the cohort of normal controls. The serum BDNF levels of the patients with ischemic heart disease did not differ depending on anamneses of diabetes mellitus, hyperlipemia, hypertension, and smoking, and no direct relationship thereof with a blood sugar level, glycohemoglobin level, serum total cholesterol level, serum LDL cholesterol level, and blood pressure was observed. Furthermore, the serum BDNF level was unrelated to the subjective symptoms of angina pectoris based on CCS classification. Considering all the factors involved, the decreased serum BDNF level corresponds to the pathophysiology of ischemic heart disease.

The main pathophysiology of ischemic heart disease is impaired blood flow in the coronary artery caused by arteriosclerosis. The patients with ischemic heart disease in the test exhibited, in all the cases, significant narrowing or clogging in the coronary artery caused by arteriosclerosis. Decrease in serum BDNF level adversely affects glucose metabolism, lipid metabolism, and the like, and however, in the test, could not be predicted from anamneses of diseases serving as other arteriosclerosis risk factors or from previously known risk factors such as blood sugar levels, glycohemoglobin, serum total cholesterol, and serum LDL cholesterol.

Thus, the present test demonstrates that BDNF measurement in blood is useful as a biological diagnostic marker for an ischemic heart disease risk group because BDNF plays a quite important role in the pathophysiology of ischemicheart disease and can particularly find patients overlooked by previously known risk factors (diagnostic markers).

### Example 2

### Procedures

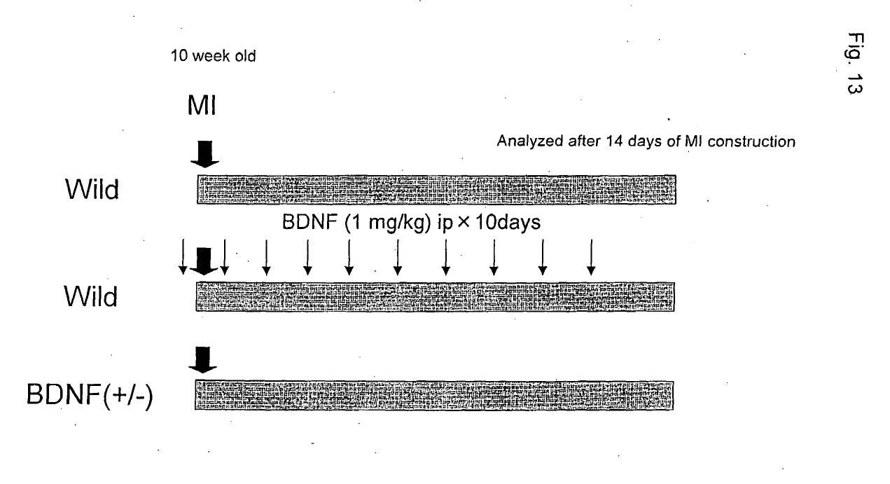

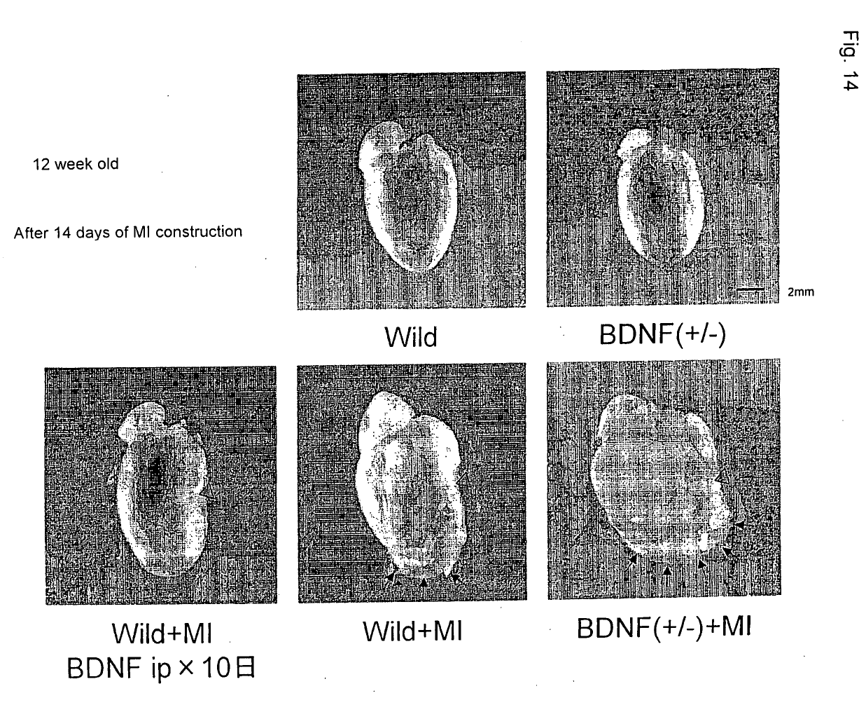

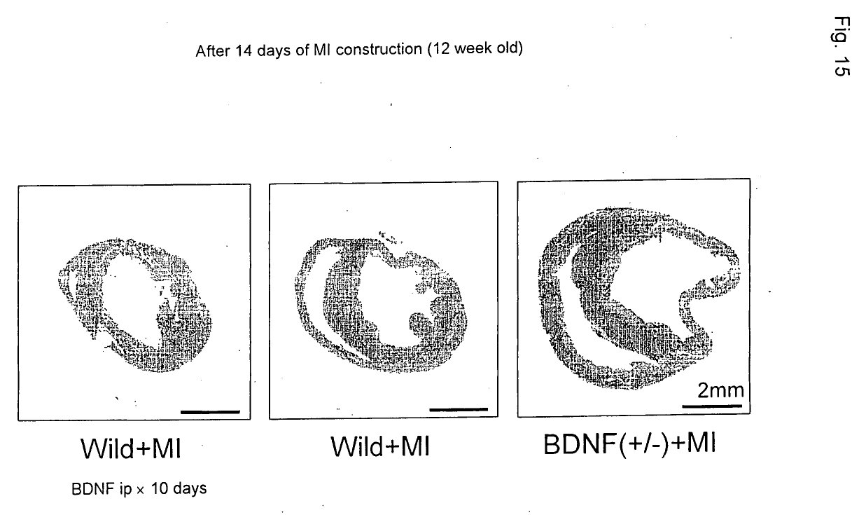

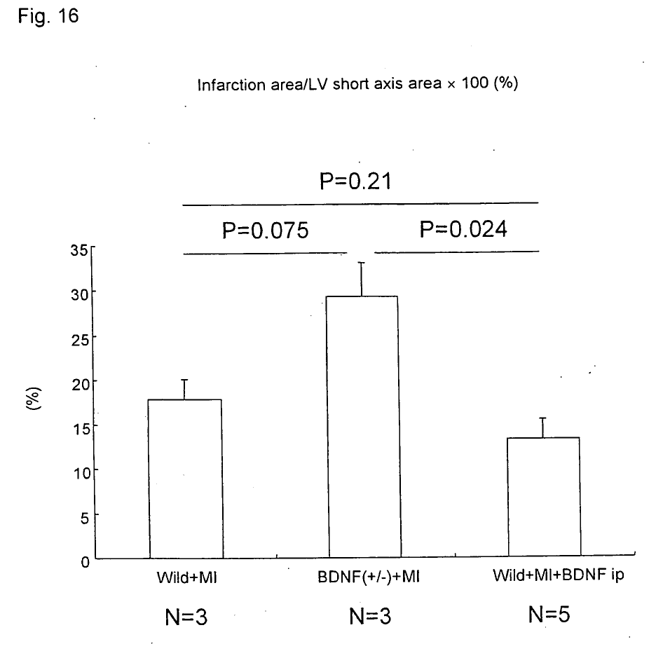

In an experiment, 10-week-old wild-type mice of C57/BL6 background (Wild) and heterozygous BDNF-knockout mice (BDNF (+/−)) (Nature (1994) 368: 147-150, obtained from THE JACKSON LABORATORY) were used. Acute myocardial infarction (MI) was constructed in these two types of mice by opening the chest under anesthesia and artificial respiration control and then ligating the left anterior descending branch of the coronary artery. They were respectively used as a “Wild+MI” group and a “BDNF(+/−)+MI” group. At the same time, the two types of mice were separately subjected to sham surgery and used as “sham” groups serving as controls. The administration of BDNF (Sumitomo Pharmaceuticals) (1 mg/kg) was initiated immediately after the construction of myocardial infarction and intraperitoneally performed for 10 consecutive days (FIG. 13). After 2 weeks of the construction of myocardial infarction, echocardiography (Agilent Sonos 4500) was performed (Table 3). Then, the mice were sacrificed, and macroscopic findings on the hearts were imaged with a stereoscopic microscope (FIG. 14). Left ventricle weights thereof were measured (Table 2). The heart samples were fixed with formalin to create paraffin slices thereof. Masson trichrome stain (FIG. 15) was used to quantify the size of myocardial infarction (FIG. 16). The difference among multiple groups was analyzed by one-way analysis of variance (ANOVA).

### (Result)

In FIG. 14, the symbol “Δ” represents the ligated left anterior descending branch of the coronary artery, and the symbol “↑” represents a site at which post-infarction myocardial remodeling was observed. Post-infarction myocardial remodeling was increased in the BDNF-knockout mouse group (BDNF(+/−) +MI) (lower right column) as compared with the wild-type mouse group with myocardial infarction (Wild+MI) (lower middle column), whereas it tended to be small in the BDNF-administered group (Wild+MI+BDNF ip) (lower left column) (FIG. 14).

Table 2 shows change in left ventricle weight of each of the test groups. The left ventricle weight (HW/BW) corrected by the body weight after myocardial infarction was significantly increased (p<0.01) in the myocardial infarction (MI) group as compared with the sham group as a control, and this increase tended to be small in the BDNF-administered group (MI+BDNF ip) (Table 2, the column indicated by “Wild”).

Increase in left ventricle weight after myocardial infarction was significantly large (p<0.05) in the BDNF-knockout mouse group with myocardial infarction (BDNF(+/−)+MI) as compared with the wild-type mouse group with myocardial infarction (Wild+MI).

FIG. 15 shows the size of myocardial infarction in each of the myocardial infarction groups. The size of infarction tended to be large in the BDNF-knockout mouse group with myocardial infarction (BDNF(+/−)+MI) as compared with the wild-type mouse group with myocardial infarction (Wild+MI) (FIGS. 15 to 16). The size of infarction tended to be small in the BDNF-administered group (Wild+MI+BDNF ip) as compared with the wild-type mouse group with myocardial infarction (Wild+MI).

Table 3 shows an analysis result by echocardiography. Significant increase in left ventricular diastolic diameter (LVDD) and significant decrease in left ventricular fractional shortening (FS) were observed in the myocardial infarction (MI) groups as compared with the sham groups as controls. Increase in left ventricular diastolic diameter (LVDD) and decrease in left ventricular fractional shortening (FS) after myocardial infarction were significantly suppressed in the BDNF-administered group (Wild+MI+BDNF ip). By contrast, a more increasing trend in left ventricular diastolic diameter (LVDD) and a more decreasing trend in left ventricular fractional shortening (FS) after myocardial infarction were exhibited in the BDNF-knockout mouse group with myocardial infarction (BDNF(+/−)+MI) as compared with the wild-type mouse group with myocardial infarction (Wild+MI).

TABLE 2
Left ventricle weight
After 14 days of MI construction (12 week old)

| | Wild | | | BDNF(+/−) | |
|---|---|---|---|---|---|
| | sham | MI | MI + BDNF ip | sham | MI |
| | M = 6 | M = 3 | M = 1 | M = 5 | M = 4 |
| n | F = 3 | F = 3 | F = 3 | F = 2 | F = 5 |
| Body weight (g) | 21.7 ± 0.67 | 20.1 ± 1.03* | 17.53 ± 1.46 | 24.61 ± 1.32 | 20.44 ± 1.05# |
| (BW) | | | | | |
| Heart weight (mg) | 108.11 ± 4.56 | 127 ± 6.20* | 109.25 ± 12.58 | 108.57 ± 4.71 | 152.44 ± 13.01# [symbol] |
| (HW) | | | | | |
| HW/BW | 4.98 ± 0.12 | 6.35 ± 0.28** | 6.20 ± 0.25** | 4.46 ± 0.19 | 7.57 ± 0.67## [symbol] |

**P < 0.01 vs Wild + sham
*P < 0.05 vs Wild + sham [symbol]
P < 0.05 vs Wild + MI
##P < 0.01 vs BDNF(+/−) + sham
#P < 0.05 vs BDNF(+/−) + sham
M: male
F: female

TABLE 3
Findings by echocardiography
After 14 days of MI construction (12 week old)

| | Wild | | | BDNF(+/−) | |
|---|---|---|---|---|---|
| | sham | MI | MI + BDNF ip | sham | MI |
| | M = 3 | M = 3 | M = 1 | M = 3 | M = 4 |
| n | F = 3 | F = 3 | F = 3 | F = 3 | F = 4 |
| LVDD (mm) | 3.63 ± 0.15 | 4.66 ± 0.19** | 3.75 ± 1.97 [symbol] | 3.59 ± 0.17 | 4.83 ± 0.16## |
| FS (%) | 39.2 ± 2.47 | 22.12 ± 2.41** | 30.53 ± 0.43* [symbol] | 40.23 ± 1.95 | 18.43 ± 1.8## |

**P < 0.01 vs Wild + sham
*P < 0.05 vs Wild + sham [symbol]
P < 0.05 vs Wild + MI
##P < 0.01 vs BDNF(+/−) + sham
#P < 0.05 vs BDNF(+/−) + sham
LVDD: left ventricular diastolic diameter
FS: left ventricular fractional shortening
M: male
F: female

It is known that after acute myocardial infarction, cardiac failure occurs due to the progression of left ventricular remodeling and reduced cardiac function associated therewith. Increase in left ventricle weight, increase in infarction size, increase in left ventricular diastolic diameter (LVDD), and decrease in left ventricular fractional shortening (FS) after myocardial infarction were observed in the mice with decreased BDNF (BDNF(+/−)) as compared with the mice with BDNF not decreased (Wild). Therefore, endogenous BDNF is considered to suppressively work for left ventricular remodeling after myocardial infarction. All of these changes are suppressed in the wild-type mouse group to which BDNF was administered after myocardial infarction (Wild+MI+BDNF ip). Therefore, the usefulness of BDNF in the suppression of post-infarction myocardial remodeling was demonstrated. Considering that BDNF expression is decreased in patients with ischemic heart disease, the administration of BDNF or a drug increasing the expression/activity thereof is quite effective as treatment in the acute stage of myocardial infarction.

1. A diagnostic agent for an ischemic heart disease risk group comprising an anti-brain-derived neurotrophic factor antibody.

2. The diagnostic agent for an ischemic heart disease risk group according to claim 1, wherein the diagnostic agent is intended for the measurement of a brain-derived neurotrophic factor concentration in blood.

3. The diagnostic agent for an ischemic heart disease risk group according to claim 1 or 2, wherein the diagnostic agent comprises an anti-brain-derived neurotrophic factor antibody and a labeled anti-brain-derived neurotrophic factor antibody.

4. A diagnostic kit for an ischemic heart disease risk group comprising an anti-brain-derived neurotrophic factor antibody and a labeling agent.

5. The diagnostic kit for an ischemic heart disease risk group according to claim 4, wherein the diagnostic kit is intended for the measurement of a brain-derived neurotrophic factor concentration in blood.

6. The diagnostic kit for an ischemic heart disease risk group according to claim 4 or 5, wherein the diagnostic kit comprises an anti-brain-derived neurotrophic factor antibody and a labeled anti-brain-derived neurotrophic factor antibody.

7. An assay method for an ischemic heart disease risk group, characterized by comprising measuring a brain-derived neurotrophic factor concentration in blood.

8. The assay method for an ischemic heart disease risk group according to claim 1, wherein the assay method measures a brain-derived neurotrophic factor concentration by use of an anti-brain-derived neurotrophic factor antibody.

9. The assay method for an ischemic heart disease risk group according to claim 7, wherein the assay method measures a brain-derived neurotrophic factor concentration by use of an anti-brain-derived neurotrophic factor antibody and a labeled anti-brain-derived neurotrophic factor antibody.

10. An assay method for a therapeutic drug for ischemic heart disease, characterized by comprising measuring a brain-derived neurotrophic factor concentration in blood.

11. A therapeutic drug for ischemicheart disease comprising a compound increasing a brain-derived neurotrophic factor.

12. A therapeutic drug for ischemic heart disease comprising a brain-derived neurotrophic factor.

13. Use of a compound increasing a brain-derived neurotrophic factor for the production of a therapeutic drug for ischemic heart disease.

14. Use of a brain-derived neurotrophic factor for the production of a therapeutic drug for ischemic heart disease.

15. A therapeutic method for ischemic heart disease, characterized by comprising administering a compound increasing a brain-derived neurotrophic factor.

16. A therapeutic method for ischemic heart disease, characterized by comprising administering a brain-derived neurotrophic factor.

17. A suppressive/preventive drug for post-infarction myocardial remodeling comprising a brain-derived neurotrophic factor.

18. Use of a brain-derived neurotrophic factor for the production of a suppressive/preventive drug for post-infarction myocardial remodeling.

19. A suppressive/preventive method for post-infarction myocardial remodeling, characterized by comprising administering a brain-derived neurotrophic factor.
